(19) United States
(12) Patent Application Publication
(10) Pub. No.: US 2025/0177291 A1
(43) Pub. Date: Jun. 5, 2025

(54) HYPOTONIC GEL-FORMING FORMULATIONS WITH ENHANCED RHEOLOGICAL PROPERTIES

(71) Applicant: The Johns Hopkins University, Baltimore, MD (US)

(72) Inventors: Laura Ensign, Towson, MD (US); Tung Heng Henry Hsueh, Baltimore, MD (US); Justin Hanes, Baltimore, MD (US)

(21) Appl. No.: 19/100,455

(22) PCT Filed: Nov. 29, 2022
(86) PCT No.: PCT/US2022/080579

§ 371 Date: Jan. 31, 2025

Related U.S. Application Data
(60) Provisional application No. 63/287,415, filed on Dec. 8, 2021.

(51) Int. Cl. A61K 9/00; A61K 9/06; A61K 45/00; A61K 47/10

(52) U.S. Cl. CPC A61K 9/0048; A61K 9/06; A61K 45/00; A61K 47/10

(57) ABSTRACT

Improved hypotonic gelling vehicles are described as solubilizing agents for drugs and as a means to provide sustained drug delivery to the eye. It has been discovered that the addition of small amounts of low molecular weight PEG to hypotonic gel-forming compositions enhances the viscosity of the solution at the ocular surface, prologs tear break-up time and increases ocular comfort. Solubilizing drugs at higher concentrations enhances drug penetration into the tissues of the body, while the hypotonic gelling vehicle further improves distribution of the drug over a larger surface area for increased absorption and sustained release for reduced side effects and longer duration of action.

### CROSS-REFERENCE TO RELATED APPLICATIONS

This application claims priority to U.S. Ser. No. 63/287,415, filed Dec. 8, 2021 by Laura Ensign, Tung Heng Henry Hsueh and Justin Hanes for “Hypotonic Gel-forming Formulations with Enhanced Rheological Properties”, the disclosure of which is hereby incorporated herein by reference in their entirety.

### STATEMENT REGARDING FEDERALLY SPONSORED RESEARCH OR DEVELOPMENT

This invention was made with government support under Grant No. EY031041 awarded by the National Institutes of Health. The government has certain rights in the invention.

### FIELD OF THE INVENTION

This invention is generally in the field of formulations for enhanced drug delivery, in particular drug delivery at epithelial surfaces such as the surface of the eye.

### BACKGROUND OF THE INVENTION

The eye is a very complex structure, with unique anatomy and physiology. The anatomy of the interior of the eye includes the anterior (front) segment, which occupies approximately one third of the total area of the eye, and posterior (rear) segment, which constitutes the remaining two thirds of the area. Tissues such as the cornea, conjunctiva, aqueous humor, iris, ciliary body, and lens make up the anterior portion, whilst the posterior segment includes the sclera, choroid, retinal pigment epithelium, neural retina, optic nerve, and vitreous humor.

Both the anterior and posterior segments of the eye are affected by various ophthalmic diseases and disorders, many of which are vision-threatening, including glaucoma, allergic conjunctivitis, anterior uveitis and cataract, age-related macular degeneration (AMD), and diabetic retinopathy. Whilst many therapeutic agents for treating ophthalmic disease are available, the delivery of effective amounts of drugs to the interior of the eye often requires uncomfortable and potentially dangerous invasive techniques, such as intra-ocular injection.

Sustained delivery of drugs through the mucosal surfaces of the eye has potential for improving the treatment and prevention of many ocular diseases, including infections, inflammatory disease, and degenerative eye conditions. However, achieving sustained prophylactic or therapeutic drug concentrations using traditional soluble dosage forms remains challenging due to degradation, rapid shedding, and rapid systemic absorption of drug.

One mechanism for the delivery of therapeutic agents to the eye is by application of topical agents onto the ocular surface, for example, by formation of an artificial tear film. The tear film (the liquid layer bathing the cornea and conjunctiva) is responsible for ocular surface comfort, mechanical, environmental, and immune protection, epithelial health; and it forms smooth refractive surface for vision. Tears prevent dryness by coating the surface of the eye, as well as protecting it from external irritants. There are no blood vessels on the surface of the eye, so oxygen and nutrients are transported to the surface cells by tears. In addition, foreign bodies that enter the eye are washed out by tears.

Recent approaches to drug delivery to the anterior segment include modulation of conventional topical solutions with permeation and viscosity enhancers, as well as development of conventional topical formulations such as suspensions, emulsions, and ointments. The use of gels that coat the eye for drug delivery has also been envisioned (see, for example, U.S. publication Nos. 2021/0196837 and 2021/0177751). Various nano-formulations have also been introduced for anterior segment ocular drug delivery, and drug releasing devices and nano-formulations are being developed for posterior ocular delivery, for example, for treating chronic vitreoretinal diseases. However, many topical formulations have poor rheological properties, such as high tackiness, and poor viscosity, leading to discomfort and disruption of the gel upon blinking.

There remains an urgent need for enhanced delivery systems that provide effective drug delivery to epithelial cells through mucosal surfaces without invasive, vision-disruptive, or uncomfortable procedures, particularly for the repeated or prolonged treatment of chronic diseases and disorders. Also, there is an unmet need for compositions for mucosal delivery that offer retention and sustained release of prophylactic, therapeutic or diagnostic agents at mucosal surfaces.

Therefore, it is an object of the present invention to provide drug delivery compositions with reduced tackiness, for increased comfort and lubrication of the eye.

It is also an object of the invention to provide compositions for delivery of active agents to the eye that provide a physical barrier to pathogen entry.

It is a further object of the present invention to provide improved compositions for delivery to epithelial tissues through mucosal surfaces, which allow retention and sustained release of prophylactic, therapeutic or diagnostic agents to the epithelial tissues via mucosal surfaces.

### SUMMARY OF THE INVENTION

It has been discovered that the addition of small amounts of low molecular weight polyethylene glycol (“PEG”) to hypotonic gel-forming compositions enhances the viscosity of the solution at the ocular surface, prolongs tear break-up time and increases ocular comfort. Compositions having improved properties for the effective delivery of drugs to epithelial tissue having mucosal surfaces, especially to the eye for treating ophthalmic diseases and disorders, have been developed. The compositions include hypotonic gel-forming polymers in combination with low molecular weight PEG, having reduced adhesion to the ocular surface and low-tackiness, while maintaining a low viscosity required for application to the eyes in the form of eye-drops. The formulation includes therapeutic, prophylactic, nutraceutical, and/or diagnostic agent, a gel-forming polymer for application to a mucosal or epithelial surface formulated so that it is at a concentration below the critical gel concentration (CGC) of the polymer under isotonic conditions and a temperature between room temperature and body temperature (about 25 to about 37° C.), a low-molecular weight PEG in an amount between about 0.1% and about 1.0% inclusive, weight/volume (w/v) of the total, and excipients to form a pharmaceutically acceptable hypotonic formulation of the polymer suitable for delivery to the mucosal or epithelial surface of an individual in need thereof.

Preferably, the gel-forming polymer is between greater than 10% and less than 18% in an aqueous excipient. In preferred embodiments, the gel-forming polymer is a poloxamer, for example, poly(ethylene glycol)-block-poly(propylene glycol)-block-poly(ethylene glycol) (PLURONIC® F127). In some embodiments, the gel-forming polymer is between 10% and 16% PLURONIC® F127. Preferred PEGs are PEG 100, PEG 200, PEG 300, PEG 400, PEG 600, PEG 800, PEG 1,000, PEG 2,000, most preferably, PEG 400. In preferred embodiments, the PEG is in an amount between about 0.01% w/v and about 1% w/v, inclusive, preferably, 0.4% w/v.

In some embodiments, the therapeutic, prophylactic, nutraceutical, or diagnostic agent to be delivered is water-soluble. In other embodiments, the therapeutic, prophylactic, nutraceutical, or diagnostic agent to be delivered is poorly water-soluble. In some embodiments, the agent is a protein or peptide, small molecule, sugar or polysaccharide, lipid, glycolipid, glycoprotein, nucleic acid, oligomer or polymer thereof, or small molecule. Preferred agents for delivery include steroids, glaucoma agents, tyrosine kinase inhibitors, immunosuppressive agents, anti-fibrotic agents, anti-infectives, hormones or chemotherapeutic agents.

The formulation releases the therapeutic, prophylactic, or diagnostic agent at the mucosal or epithelial surface over a period of at least one hour, preferably at least 24 hours. Preferably, the gel-forming polymer forms a uniformly thick layer at the time of administration onto the mucosal or epithelial surface.

Exemplary mucosal or epithelial surfaces include ocular, oral, pharyngeal, esophageal, pulmonary, aural, nasal, buccal, lingual, vaginal, cervical, genitourinary, alimentary, and anorectal surfaces. In preferred embodiments, the mucosal or epithelial surface is the ocular surface of the eye, and the formulation retains an effective concentration of the therapeutic, prophylactic, or diagnostic agent at the epithelial tissues inside the eye for more than a week, for example, in any one of the epithelial tissues such as cornea, aqueous humor, sclera, conjunctiva, iris, lens, retina, and retinal pigment epithelium.

Generally, the formulation for administration in the form of a dry powder, gel, or liquid. In some embodiments, the formulation is provided in a single or multiple dosage unit for administration.

Methods of administering the hypotonic gel-forming formulations to an epithelial surface, preferably a mucosal surface, or in need thereof are also described. The methods are particularly suited for administering to an ocular surface of the eye to treat or prevent one or more diseases or disorders of the eye. Exemplary diseases or disorders include glaucoma, dry eye syndrome (DES), macular degeneration, diabetic retinopathy, scleroderma, and cancer. When administered to the eye, the formulation is administered as an eye drop into the eye of the subject, preferably in an amount between about 10 μl and about 100 μl, inclusive, of the formulation. In some embodiments, the method is repeated once or more as needed, for example, hourly, daily, every other day, every three days, every four days, every five days, every six days, weekly, every two weeks, or less often. The formulation typically forms a gel at the surface of the eye having a thickness of between about 0.01 mm and 2 mm, inclusive, or between about 0.5 mm and 1.5 mm, inclusive. In some embodiments, the formulation prior to forming a gel at the surface of the eye has a tonicity of between about 50 mOsm/L and about 280 mOsm/L, inclusive. In some embodiments, the gel formed form the formulation has a tonicity close to isotonic after the water absorption occurs and equilibrium is re-established.

### BRIEF DESCRIPTION OF THE DRAWINGS

FIGS. 1A-1B are bar graphs of rheological properties of 16% (w/v) PLURONIC® F127 with 0-1% glycerin added, showing Adhesion (0-1.2 J/m²) (FIG. 1A) and Max Tack (0-1.0 N) (FIG. 1B) (n=3-6).

FIGS. 2A-2B are bar graphs of rheological properties of 16% (w/v) PLURONIC® F127 with 0-1% PEG 300 added, showing Adhesion (0-1.2 J/m²) (FIG. 2A) and Max Tack (0-1.0 N) (FIG. 2B) (n=3-6), *p<0.05.

FIGS. 3A-3B are bar graphs of rheological properties of 16% (w/v) PLURONIC® F127 with 0-1% PEG 400 added, showing Adhesion (0-1.2 J/m²) (FIG. 3A) and Max Tack (0-0.8 N) (FIG. 3B) (n=3-6), *p<0.05.

FIGS. 4A-4B are bar graphs of rheological properties, showing Adhesion (0-1.2 J/m²) (FIG. 4A) and Max Tack (0-1.0 N) (FIG. 4B) of 16% (w/v) PLURONIC® F127 with No additive, or the following additives: 0.4% PEG 300, 0.4% PEG 400, 0.4% PEG 400+0.3% polyglycerol (“PG”), Dextran 70, 1% carboxymethyl cellulose (“CMC”), 0.5% Polyoxyethylene (20) sorbitan monooleate (“PS80”) and 0.4% PG samples, respectively. (n=3-6), *p<0.05.

FIG. 5 is a line graph showing Viscosity (10¹-10⁵ mPa·s) over Temperature (15-42° C.) for each of 16% PLURONIC® F127 ( ), and 16% PLURONIC® F127 +0.4% PEG 400 ( ), respectively.

FIGS. 6A-6B are bar graphs of the force required to expel a first droplet from a bottle, showing Squeeze Force (0-20 N) for samples including Saline, 12% PLURONIC® F127, 12% F127 +0.3% PEG400, and 18% PLURONIC® F127, respectively, at room temperature (FIG. 6A), and 33° C. (FIG. 6B), respectively. *At 33° C., the 18% F127 formed a gel and was extruded as a band of gel rather than a droplet.

FIG. 7 is a bar graph of the force required to expel a first droplet from a bottle, showing Squeeze Force (0-25 N) for samples including Saline, 12% PLURONIC® F127, 12% PLURONIC® F127 +0.3% PEG 300, 12% PLURONIC® F127 +0.3% PEG 400, BROMSITE® (“bromfenac ophthalmic solution, 0.075%”), and SYSTANE® Ultra (Alcon lubricating eye drops, where the active ingredients are polyethylene glycol 400 (0.04%) and propylene glycol (0.3%), respectively). * bromfenac ophthalmic solution, 0.075% (“BROMSITE®”) required a significantly higher squeeze force than saline (p<0.05).

FIG. 8 is a bar graph of Tear break up time (TBUT), as measured in the eyes of New Zealand White rabbits, showing TBUT (0-60 Sec) for each sample including Saline, SYSTANE®, GENTEAL® (lubricating eyedrops containing carboxymethylcellulose, dextran, glycerin, hypromellose, polyethylene glycol 400 (PEG 400), polysorbate, polyvinyl alcohol, povidone, or propylene glycol), and 12% F127+0.4% PEG400, respectively. n=4 different eyes per group with two independent measurements made by masked observers; *p<0.05 compared to saline, #p<0.05 compared to all other formulations.

FIGS. 9A-9H are graphs of OcuGel without preservative (n=3) stability at room temperature for up to 6 months. Measurements of (9A) pH, (9B) absorbance (AU), (9C) viscosity (mPa*s, shear rate 100 s−1), (9D) osmolality (mOsm/kg), (9E) BAK concentration (% w/v), and Poloxamer 407 molecular weight (Mw) (9F), polydispersity (9G) and % w/v in the OcuGel formulation (9H).

FIGS. 10A-10G are graphs of OcuGel (n=3) stability at room temperature for up to 3 months. Measurements of (10A) pH, (10B) absorbance (AU), (10C) viscosity (mPa*s, shear rate 100 s−1), (10D) osmolality (mOsm/kg), and Poloxamer 407 (10E) molecular weight (Mw), (10F) polydispersity and (10G) % w/v in the formulation.

FIG. 11 is a graph of OcuGel (12% Poloxamer 407, 0.4% PEG400, 1 mM borate buffer, 0.01% BAK) with or without the addition of 0.2% HA at various osmolality (72-300 mOsm/kg) dosed to NZW rabbits, measured as Fluorescein-based tear break up time (TBUT) at 90 min after dosing, compared to no treatment, saline, and SYSTANE® HYDRATION PF. *p<0.05 compared to OcuGel at 72 mOsm/kg.

### DETAILED DESCRIPTION OF THE INVENTION

### I. Definitions

As generally used herein “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

“Biocompatible” and “biologically compatible”, as used herein, generally refer to materials that are, along with any metabolites or degradation products thereof, generally non-toxic to the recipient, and do not cause any significant adverse effects to the recipient. Generally speaking, biocompatible materials are materials which do not elicit a significant inflammatory, immune or toxic response when administered to an individual.

The terms “gel” and “hydrogel”, as used herein, refers to a swollen, water-containing network of finely dispersed polymer chains that are water-insoluble, where the polymeric molecules are in the external or dispersion phase and water (or an aqueous solution) forms the internal or dispersed phase. The chains can be chemically crosslinked (chemical gels) or physically crosslinked (physical gels). Chemical gels possess polymer chains that are connected through covalent bonds, whereas physical gels have polymer chains linked by non-covalent bonds or cohesion forces, such as Van der Waals interactions, ionic interaction, hydrogen bonding, or hydrophobic interaction.

The polymer chains are typically hydrophilic or contain hydrophilic polymer blocks. “Gel-forming polymers” is used to describe any biocompatible polymer, including homopolymers, copolymers, and combinations thereof, capable of forming a physical hydrogel in an aqueous medium when present at or above the critical gel concentration (CGC).

The “critical gel concentration”, or “CGC”, as used herein, refers to the minimum concentration of gel-forming polymer needed for gel formation, e.g., at which a solution-to-gel (sol-gel) transition occurs. The critical gel concentration can be dependent on a number of factors, including the specific polymer composition, molecular weight, temperature, and/or the presence of other polymers or excipients.

The term “thermosensitive gel-forming polymer” refers to a gel-forming polymer that exhibits one or more property changes with a change in the temperature. For example, some thermosensitive gel-forming polymers are water soluble below a certain temperature but become water insoluble as temperature is increased. The term “lower critical solution temperature (LCST)” refers to a temperature, below which a gel-forming polymer and solvent are completely miscible and form a single phase. For example, “the LCST of a polymer solution” means that the polymer is uniformly dispersed in a solution at that temperature (i.e., LCST) or lower, but aggregates and forms a second phase when the solution temperature is increased beyond the LCST.

“Hydrophilic,” as used herein, refers to molecules which have a greater affinity for, and thus solubility in, water as compared to organic solvents. The hydrophilicity of a compound can be quantified by measuring its partition coefficient between water (or a buffered aqueous solution) and a water-immiscible organic solvent, such as octanol, ethyl acetate, methylene chloride, or methyl tert-butyl ether. If after equilibration a greater concentration of the compound is present in the water than in the organic solvent, then the compound is considered hydrophilic.

“Hydrophobic,” as used herein, refers to molecules which have a greater affinity for, and thus solubility in, organic solvents as compared to water. The hydrophobicity of a compound can be quantified by measuring its partition coefficient between water (or a buffered aqueous solution) and a water-immiscible organic solvent, such as octanol, ethyl acetate, methylene chloride, or methyl tert-butyl ether. If after equilibration a greater concentration of the compound is present in the organic solvent than in the water, then the compound is considered hydrophobic.

As used herein, “ocugel” refers to 12% F127, 0.4% PEG400 and 1 mM borate buffer, formulated with or without a preservative such as 0.01% benzalkonium chloride (“BAK”), at either 72 mOsm or 150 mOsm.

As used herein, the term “treating” includes inhibiting, alleviating, preventing, or eliminating one or more symptoms or side effects associated with the disease, condition, or disorder being treated.

The term “reduce”, “inhibit”, “alleviate” or “decrease” are used relative to a control, either no other treatment or treatment with a known degree of efficacy. One of skill in the art would readily identify the appropriate control to use for each experiment. For example, a decreased response in a subject or cell treated with a compound is compared to a response in subject or cell that is not treated with the compound.

As used herein the term “effective amount” or “therapeutically effective amount” means a dosage sufficient to treat, inhibit, or alleviate one or more symptoms of a disease state being treated or to otherwise provide a desired pharmacologic and/or physiologic effect. The precise dosage will vary according to a variety of factors such as subject-dependent variables (e.g., age, immune system health, etc.), the disease or disorder, and the treatment being administered. The effective amount can be relative to a control. Such controls are known in the art and discussed herein, and can be, for example the condition of the subject prior to or in the absence of administration of the drug, or drug combination, or in the case of drug combinations, the effect of the combination can be compared to the effect of administration of only one of the drugs.

“Excipient” is used herein to include a compound that is not a therapeutically or biologically active compound. As such, an excipient should be pharmaceutically or biologically acceptable or relevant, for example, an excipient should generally be non-toxic to the subject. “Excipient” includes a single such compound and is also intended to include a plurality of compounds.

The term “osmolarity”, as generally used herein, refers to the total number of dissolved components per liter. Osmolarity is similar to molarity but includes the total number of moles of dissolved species in solution. An osmolarity of 1 Osm/L means there is 1 mole of dissolved components per L of solution. Some solutes, such as ionic solutes that dissociate in solution, will contribute more than 1 mole of dissolved components per mole of solute in the solution. For example, NaCl dissociates into Na⁺ and Cl⁻ in solution and thus provides 2 moles of dissolved components per 1 mole of dissolved NaCl in solution. Physiological osmolarity is typically in the range of about 280 to about 310 mOsm/L.

The term “tonicity”, as generally used herein, refers to the osmotic pressure gradient resulting from the separation of two solutions by a semi-permeable membrane. In particular, tonicity is used to describe the osmotic pressure created across a cell membrane when a cell is exposed to an external solution. Solutes that can cross the cellular membrane do not contribute to the final osmotic pressure gradient. Only those dissolved species that do not cross the cell membrane will contribute to osmotic pressure differences and thus tonicity. The term “hypertonic”, as generally used herein, refers to a solution with a higher concentration of solutes than is present on the inside of the cell. When a cell is immersed into a hypertonic solution, the tendency is for water to flow out of the cell in order to balance the concentration of the solutes. The term “hypotonic”, as generally used herein, refers to a solution with a lower concentration of solutes than is present on the inside of the cell. When a cell is immersed into a hypotonic solution, water flows into the cell in order to balance the concentration of the solutes. The term “isotonic”, as generally used herein, refers to a solution wherein the osmotic pressure gradient across the cell membrane is essentially balanced. An isotonic formulation is one which has essentially the same osmotic pressure as human blood. Isotonic formulations will generally have an osmotic pressure from about 250 to 350 mOsm.

“Gel-forming concentration” refers to the concentration of the polymer at which it undergoes a phase shift from a solution to a gel. Critical gelation concentration (CGC) corresponds to the minimum gelator concentration required for gelation.

### II. Enhanced Hypotonic Gel-Forming Compositions

Hypotonic formulations of hydrogel forming polymers, preferably comprising poloxamers, having improved rheological properties, have been developed for enhanced delivery of therapeutic, diagnostic, prophylactic, or other agents, to epithelial tissues. The formulations are particularly suited for enhanced delivery of therapeutic, diagnostic, prophylactic, or other agents to the eye. Other suitable epithelial tissues include oral surfaces, pharyngeal surfaces, esophageal surfaces, pulmonary surfaces, ocular surfaces, aural surfaces, nasal surfaces, buccal surfaces, lingual surfaces, vaginal surfaces, cervical surfaces, genitourinary surfaces, alimentary surfaces, anorectal surfaces, and/or skin surfaces. In preferred embodiments, the epithelial tissues or surfaces are vaginal and rectal surfaces. In many of these tissues, the epithelial cells underlie a mucosal coating.

The polymers are administered at a concentration less than their normal critical gelling concentration (CGC). A poloxamer gel administered onto the surface of a mucosal surface such as the eye at a concentration equal to or above its CGC will assemble into a bolus on the surface. On the ocular surface, a bolus of gel is rapidly cleared away by blinking. In contrast, fluid from a hypotonically-administered poloxamer solution, where the poloxamer is at a concentration below its CGC, will form a uniform, thin gel coat across the entire surface of the eye, thereby maintaining a reservoir of drug in close association with the mucosal surface and enhancing and facilitating delivery of agents to the eye. The presence of additional low molecular weight polyoxyethylene oxide polymer such as PEG 300 or PEG400 reduces adhesion to the ocular surface, improving the comfort and lubrication of the eye and providing enhanced resistance to disruption by blinking and tearing. As water is absorbed into the tissues, the poloxamer becomes concentrated and forms a gel near the epithelial tissue surface, thereby trapping drug molecules in a sustained-release gel on the tissue surface (rather than, e.g., in a bolus of gel that forms primarily in the lumen as occurs with traditional thermogelling methods whereby the gelling polymers are administered at a concentration at or above their CGC). Endogenous mucin glycopolymers affect the gelling properties of the hypotonic gelling agents, including the concentration of gelling agent needed to gel and the pore structure of the resulting gel/mucin mixture. After mucosal application, the hypotonic gelling vehicles coat the epithelium, including the folds or inner eyelids.

The examples demonstrate that the addition of a polymer such as low molecular weight polyethylene glycols (e.g., PEG300 or PEG400 at around 0.1%-1.0% w/w), but not high molecular weight PEG (1000 D or larger or 2000 D or larger), reduces adhesion of gels to the mucosal surface of the eye. The reduction in adhesion prolongs the tear break-up time (retention) of the formulation administered to the eye while maintaining viscosity, compared to an equivalent gel in the absence of low-molecular weight PEG. Therefore, the addition of low molecular weight PEGs to hypotonic formulations of hydrogel forming polymers for ocular administration enhances the residence time of the resulting hydrogels at the surface of the eye by providing improved flexibility and/or reduced tackiness.

Improved hypotonic formulations of hydrogel forming polymers capable of forming uniform gel coatings on epithelial surfaces with reduced tackiness, but which do not gel under storage conditions, contain one or more gel-forming polymers in a hypotonic carrier, low molecular weight PEGs, and optionally contain one or more additional excipients and/or one or more therapeutic, prophylactic, or diagnostic agents.

Typically, the hydrogel forms a thin coating that covers the entire ocular surface. The size and thickness of the coating is dependent upon the amount of the material applied and the size/shape of the ocular surface. In some embodiments the hydrogel forms a thin gel at the surface of the eye that is between about 0.01 mm and about 2 mm in thickness, inclusive, or between about 0.5 mm and 1.5 mm, inclusive; for example, about 0.01 mm, 0.05, 0.1, 0.15, 0.2, 0.3. 0.4. 0.5, 0.6. 0.7, 0., 0.9, 1.0, 1.5, or 2 mm in thickness, or more than 2 mm in thickness. In some embodiments, the gel forms a film that coats the entire ocular surface with unform thickness. In other embodiments, the gel coats the ocular surface with a film that is non-uniform in thickness, for example, having a concave or convex conformation. In some embodiments, the gel film at the ocular surface prevents pathogens from contacting the surface of the eye. For example, in some embodiments, the gel film prevents bacteria, viruses, fungi or protozoan pathogens from contacting the eye.

### A. Hydrogel-Forming Polymers

The hypotonic gel-forming compositions contain one or more gel-forming polymers. Gel-forming polymers are utilized at a concentration below the normal critical gel concentration (CGC) of the polymer, e.g., the concentration at which the polymer solution would gel in a test tube when warmed to 37° C.

Thermosensitive or thermoresponsive hydrogels form solutions that undergo sol-gel transitions when the following criteria are met:
1) at or above the critical gelling concentration (CGC), and
2) at or above the critical gelling temperature.

Thermosensitive gelling agents (at or above their CGC) used for biomedical applications are liquid at room temperature but form a gel at body temperature. The increase in temperature induces a rearrangement and alignment of the polymer chains, leading to gelation into a three-dimensional structure. This phenomenon is generally governed by the ratio of hydrophilic to hydrophobic moieties on the polymer chain. A common characteristic is the presence of a hydrophobic methyl, ethyl, or propyl group.

Thermosensitive polymers that fit these criteria can be administered topically in a hypotonic solution at a range of concentrations that is below its CGC to mucosal and/or epithelial tissues to form a uniform gel coating in vivo.

Examples of thermosensitive gel forming polymers that can be used include polyoxyethylene-polyoxypropylene-polyoxyethylene triblock copolymers such as, but not limited to, those designated by the CTFA names poloxamer 407 (CAS 9003 Nov. 6, molecular weight 9,840-14,600 g/mol, percentage of polyoxyethylene by weight approximately 70%; available from BASF as LUTROL® F127) (F127) and poloxamer 188 (CAS 9003-11-6, molecular weight 7680-9510 g/mol, percentage of polyoxyethylene by weight approximately 80%; available from BASF as LUTROL® F68); Poloxamers are also known by the trade name PLURONIC® e.g., PLURONIC® F98 (CAS 9003-11-6, molecular weight 13000 g/mol, percentage of polyoxyethylene by weight approximately 80%; available from BASF); Tetronics tetra-functional block copolymers based on ethylene oxide and propylene oxide available from BASF as TETRONIC®; poly(N,N-diethylacrylamide); poly(N,N-dimethylacrylamide); poly(N-vinylcaprolactam); poly(N-alkylacrylamide); poly(N-vinylalkylamide); poly(N-isopropyl acrylamide); polyethylene oxide methacrylate polymers; poly(lactic-co-glycolic acid) (PLGA)-polyethylene glycol triblock copolymers (PLGA-PEG-PLGA and PEG-PLGA-PEG); polycaprolactone (PCL)-polyethylene glycol triblock copolymers (PCL-PEG-PCL and PEG-PCL-PEG); chitosan; and combinations thereof.

The hydrogels can be formed from individual gel forming polymers or as a combination of gel formers. For example, a poloxamer and another gel forming polymer (e.g., a tetronic polymer) may be used in combination to attain the desired characteristics. In addition, various forms of the same gel former (e.g., Poloxamer 188 and Poloxamer 407) can be combined to attain the desired characteristics.

The polymer is provided in a concentration less than the concentration in aqueous solution that forms a gel in a test tube when heated to 37° C. The concentration must be sufficiently high, but below the CGC, for the epithelium to absorb enough fluid for the CGC to be reached in vivo, so gelation can occur preferentially on/near the mucosal epithelial surface, for example, at the surface of the eye. The range of time that it takes for gelation to occur depends on the mucosal surface (the capacity and rate of water absorption), the tonicity of the solution administered (more hypotonic solutions will drive more rapid fluid absorption), and the concentration of polymer administered (if the polymer concentration is too low, not enough fluid absorption will occur to concentrate the polymer to its CGC). Gelation generally occurs within 1-20 seconds, inclusive, upon administration onto the surface of the eye, for example, as an eyedrop. For example, in some embodiments, gelation occurs within less than a second, or within 1-2 seconds, 2-3, 3-4, 4-5, 5-6, 6-7, 7-8, 8-9, 9-10, or less than 5 seconds, or less than 10 seconds following administration onto the surface of the eye.

The concentration of the polymer and the presence of additional components such as the endogenous mucins affect coverage and rate and degree of gelling. Earlier studies have shown that 18% PLURONIC® F127 gel mixed with purified pig gastric mucins (1%) or human cervicovaginal mucus (1:1 ratio) did not trap virus-sized (˜100 nm) nanoparticles (polyethylene glycol coated polystyrene nanoparticles, PSPEG) as effectively as 18% F127 gel alone. In contrast, 24% F98 gel more effectively trapped PSPEG particles when mixed with mucins or human cervicovaginal mucus. However, in vivo viral trapping with hypotonic gelling agents was more effective at trapping viruses, including human immunodeficiency virus (HIV, ˜120 nm) and herpes simplex virus (HSV, ˜180 nm). Administration of hypotonic solution containing 18% PLURONIC® F98, having a CGC of 24%, results in effective trapping of subsequently administered HIV in the vagina. Similarly, hypotonic solution containing 10% and 15% F127, having a CGC of 18%, were also effective in decreasing the MSD of HIV, indicating trapping. Additionally, both 15% PLURONIC® F127 and 18% PLURONIC® F98 reduced the diffusion of subsequently administered HSV in mouse vaginal mucus. The distribution of the individual virus MSD at a time scale of 1 s illustrated that the trapping (shift to the left) of the viruses was more uniform in the gel formed by the hypotonic 18% PLURONIC® F98 vehicle compared to 15% PLURONIC® F127. In additional tests of viral trapping by hypotonic gelling agents in the colorectum, it was found that 12% PLURONIC® F98 (CGC 24%) did not effectively trap PSPEG nanoparticles administered 30 mins after the gelling vehicle, though 18% PLURONIC® F98 was effective at trapping PSPEG nanoparticles in the mouse colorectum. Importantly, these examples illustrate differences in the gels that form when the hypotonic gelling agents are administered to different mucosal surfaces, and in this case, mix with vaginal mucus compared to colorectal mucus prior to gelling.

Therefore, in some embodiments, the gel forming polymer is present within the formulation in an amount between 1% and 50%, for example, between 5% and 20%, inclusive, such as 5%, 6%, 7%, 8%, 9%, 10%, 11%, 12%, 13%, 14%, 15%, 16%, 17%, 18%, 19% or 20%. Typically, formulations for administration to the ocular surface include an amount of hydrogel forming polymer that is a concentration below the critical gel concentration (CGC) of the polymer under isotonic conditions and a temperature between room temperature and body temperature (from about 25° C. to about 37° C., inclusive).

A preferred hydrogel forming polymer is poly(ethylene glycol)-block-poly(propylene glycol)-block-poly(ethylene glycol) (PLURONIC® F127; “F127”). In some embodiments, formulations for administration to the eye include PLURONIC® F127 at a concentration below the CGC, for example, between 5% and 18%, inclusive, for example, between about 8% and about 15%, between about 10% and about 14%, or between about 11% and about 13%, inclusive. In a particular embodiment, the PLURONIC® F127 is present in an amount of approximately 12%.

### B. Hypotonic Carriers

The gel-forming compositions include a hypotonic carrier. The hypotonic carrier will typically be a biocompatible carrier that preferably causes little to no signs of irritation when administered to the eyes of human subjects. The carrier can be naturally occurring or non-naturally occurring including both synthetic and semi-synthetic carriers. Preferred carriers are water-based. Other solutions, including sugar-based (e.g., glucose, mannitol) solutions and various buffers (phosphate-buffers, tris-buffers, HEPES), may also be used.

When hypotonic solutions are applied to an epithelial surface, such as the surface of the eye, a fluid shift occurs, and water is moved into the epithelial tissue. This can cause swelling of the epithelial cells. In some cases, when the osmotic pressure difference is too large, the epithelial cells may burst causing tissue irritation or disruption of the epithelial membrane.

Hypotonic solution refers to a solution that causes water absorption by the epithelial surface to which it is administered. Examples of hypotonic solutions include, but are not limited to, Tris[hydroxylmethyl]-aminomethane hydrochloride (Tris HCl, 10-100 mM, pH. 6-8), (4-(2-hydroxyethyl)-1-piperazineethanesulfonic acid (HEPES, 10-100 mM, pH 6-8) and dilute solutions of PBS, such as a solution containing 0.2 grams KCl, 0.2 grams KH₂PO₄, 8 grams NaCl, and 2.16 grams Na₂HPO₄*7H₂O in 1000 ml H₂O.

Hypotonic carriers cause dissolved gel-forming polymers to concentrate at an epithelial surface, resulting in uniform gel formation on the surface. The hypotonic carrier usually contains water as the major component. The hypotonic carrier can be water, although mixtures of water and a water-miscible organic solvent can also be used. Suitable water-miscible organic solvents include alcohols, such as ethanol, isopropanol; ketones, such as acetone; ethers such as dioxane; and esters such as ethyl acetate.

Therefore, in some embodiments, the hypotonic carrier is distilled water containing one or more osmolarity modifying excipients. Sodium chloride is the excipient that is most frequently used to adjust osmolarity if a solution is hypotonic. Other excipients used to adjust hypotonic solutions include glucose, mannitol, glycerol, propylene glycol and sodium sulfate. Osmolarity modifying excipients can include pharmaceutically acceptable salts such as sodium chloride, sodium sulfate, potassium chloride, and other salts to make buffers such as dibasic sodium phosphate, monobasic potassium phosphate, calcium chloride, and magnesium sulfate. Other excipients used to adjust tonicity can include glucose, mannitol, glycerol, or propylene glycol.

In some embodiments, the hypotonic carrier has an osmolarity less than the effective isotonic point (the concentration at which fluid is neither absorbed nor secreted by the underlying tissues) at that mucosal surface. The isotonic point varies for different mucosal surfaces and different buffers, depending on active ion transport at that epithelial surface, e.g., 0.9% solution of NaCl (Normal Saline) is iso-osmotic with blood and tears. Human tear fluid has an osmolality in the range of 280-320 mOsm/L, while the osmolarity of the aqueous layer of the precorneal tear film is approximately 300 mOsm/L. Therefore, in some embodiments the solution has a tonicity at or below 280 mOsm/L, for example, from 50 mOsm/L to 280 mOsm/L, from 100 mOsm/L to 280 mOsm/L, from 150 mOsm/L to 250 mOsm/L, from 200 mOsm/L to 250 mOsm/L, from 220 mOsm/L to 250 mOsm/L, from 220 mOsm/L to 260 mOsm/L, from 220 mOsm/L to 270 mOsm/L, or from 220 mOsm/L to 280 mOsm/L.

The isotonic point in the vagina for sodium-based solutions is about 300 mOsm/L, but in the colorectum, it is about 450 mOsm/L. In some embodiments the solution is hypotonic at the mucosal surface of colorectum, having a tonicity at or below 400 mOsm/L, for example, from 50 mOsm/L to 400 mOsm/L; at or below 350 mOsm/L, for example, from 50 mOsm/L to 350 mOsm/L; or at or below 300 mOsm/L, for example, from 50 mOsm/L to 280 mOsm/L. In some embodiments, the solution is hypotonic at the mucosal surface of vagina, having a tonicity at or below 300 mOsm/L, for example, from 50 mOsm/L to 280 mOsm/L.

The hypotonic carrier can include one or more pharmaceutically acceptable acid, one or more pharmaceutically acceptable base, or salts thereof. Pharmaceutically acceptable acids include hydrobromic, hydrochloric, and sulphuric acids, and organic acids, such as methanesulphonic acids, tartaric acids, and malic acids. Pharmaceutically acceptable bases include alkali metal (e.g., sodium or potassium) and alkali earth metal (e.g., calcium or magnesium) hydroxides and organic bases such as pharmaceutically acceptable amines. The hypotonic carrier can include pharmaceutically acceptable buffers such as citrate buffers or phosphate buffers.

C. Polyoxyalkylene glycol (PEO or PEG)

The improved hypotonic gel-forming compositions contain one or more additional PEGs as shear thinning polymers. In preferred embodiments, the one or more additional PEGs are low molecular weight PEGs.

Increased viscosity under no shear leads to increased residence time on the mucosal surface of the eye, while shear thinning when blinking maximizes comfort and lubrication. Thus, gelling materials with these properties have favorable lubricating properties on the surface of the eye while resisting clearance upon blinking. In some examples, one or more low immunogenic and biocompatible polymers are incorporated into the gelling vehicle for increasing viscosity under no shear and/or shear thinning when blinking. In some examples, shear thinning polymers in the concentration range in which they conventionally demonstrate shear thinning properties are also included in the composition. The shear thinning polymers typically include small amounts (0.01%-2%, inclusive) of water-soluble, low immunogenic and biocompatible polymers. A preferred shear thinning polymer is a low-molecular weight polyethylene glycol (PEG). Exemplary PEGs have a molecular weight of from about 100 Da to about 10,000 Da, inclusive, such as PEG 200, PEG 300, PEG 400, PEG 600, PEG 800, PEG 900, and PEG 1,000. In preferred embodiments, PEGs have a molecular weight of from about 100 Da to about 5,000 Da, inclusive; from about 100 Da to about 2,000 Da, inclusive; from about 100 Da to about 1,000 Da, inclusive; from about 100 Da to about 800 Da, inclusive; and from about 200 Da to about 500 Da, inclusive. In some embodiments, one or more PEGs are present within the improved hypotonic gel-forming compositions in an amount between 0.1% and 1%, inclusive, weight: volume (w: v) of the total, for example, 0.1%, 0.2%, 0.3%, 0.4%, 0.5%, 0.6%, 0.7%, 0.8%, 0.9% and 1.0%. In a preferred embodiment, a low-molecular weight PEG is present in an amount of 0.4% weight: volume (w: v) of the total.

In some embodiments, the formulations are for use as eye drops that are expelled from a bottle or other dispenser directly onto the eye. Typically, formulations for use as eye drops include lubricating agents of a type and in an amount that does not increase the squeeze force required for the expulsion of a drop, for example, such that the eye drops may be expelled as a drop for administration to the eye directly from a bottle of solution, e.g., in the form of a drop, using a squeeze-force that is similar to that required to expel a similar-sized drop of saline solution or another isotonic solution from the same bottle. For example, in some embodiments, the squeeze force required for the expulsion of a drop is between 10 Newtons (N) and 20 N, for example, about 11 N, 12 N, 13 N, 14 N, 15 N, 16 N, 17 N, 18 N, 19 N or about 20 N. In a preferred embodiment, the squeeze force required to expel a drop of the hypotonic gel-forming formulation from the bottle is between approximately 15 N-17 N, inclusive.

The addition of low-molecular weight PEG to formulations also increases the tear break up time (TBUT) upon administration of the formulation onto the ocular surface. Generally, >10 seconds is thought to be normal TBUT in human subjects, (e.g., 10, 11, 12 seconds), 5 to 10 seconds marginal, and <5 seconds is considered low time for TBUT. A short tear break-up time is a sign of a poor tear film and the longer it takes the more stable the tear film. Therefore, in some embodiments, the addition of PEG to formulations increases the tear break up time (TBUT) by between 1 second (S) and 10 S, inclusive, as compared to the TBUT associated with administering commercially available formulation with no PEG. For example, in some embodiments, the TBUT following administration in vivo is the normal time or, about 5%, 10%, 20%, 30%, 40%, 50%, 60%, 70%, 80%, 90%, 100% greater than normal time. Normal TBUT is generally >10 s in humans. In a preferred embodiment, the TBUT is extended to between 15 and 60 seconds, between 20 and 50 seconds, between 25 and 45 seconds, between 30 and 40 seconds, inclusive, following administration of the formulation to the eye in vivo.

### D. Additional Agents

Typically, the hypotonic gel-forming compositions contain one or more agents to be delivered to the eye. Examples include therapeutic agents, prophylactic agents, and/or diagnostic agents. A biologically active agent is a substance used for the treatment (e.g., therapeutic agent), prevention (e.g., prophylactic agent), diagnosis (e.g., diagnostic agent), or to effect a cure or mitigation of disease or illness, alter the structure or function of the body, or pro-drugs, which become biologically active or more active after they have been placed in a predetermined physiological environment. These may be small-molecule drugs ((e.g., molecular weight less than 2000, 1500, 1000, 750, or 500 atomic mass units (amu)), peptides or proteins, sugars or polysaccharides, nucleotides or oligonucleotides such as aptamers, siRNA, and miRNA, lipids, glycoproteins, lipoproteins, or combinations thereof.

The agents can include one or more of those described in Martindale: The Complete Drug Reference, 37^{th} Ed. (Pharmaceutical Press, London, 2011).

In one embodiment, the agent to be delivered is poorly soluble in water, but soluble in the carrier containing the gelling polymer(s). In other embodiments, the agents are water-soluble. Data also show that benefit is obtained with water soluble drugs, for example, brimonidine tartrate, which is soluble up to approximately 2-3 mg/mL.

The hypotonic gel-forming formulations can contain a therapeutically effective amount of a therapeutic agent to treat, inhibit, or alleviate one or more symptoms of a disease state being treated. The hypotonic gel-forming compositions can contain an effective amount of a prophylactic agent to prevent one or more symptoms of a disease or disorder.

### 1. Therapeutic Agents

In some embodiments, the formulations include one or more therapeutic agents. Therapeutic agents to be delivered can include anti-infective (antibiotics, antivirals, antifungals), agents for treatment of eye disorders (glaucoma, dry eye), anti-inflammatoires, inhibitors of neovascularization and/or fibrosis, neuroactive agents, or chemotherapeutics for treatment of a disease such as aberrant neovascularization or cancer. Exemplary agents include brinzolamide, cyclosporine A, brimonidine tartrate, moxifloxacin, budesonide, sunitinib, and acriflavine.

In some embodiments, the formulations include one or more protein therapeutic agents. In preferred embodiments, the formulations include one or more short therapeutic peptides. Examples of useful proteins include hormones such as insulin, growth hormones including somatomedins, and reproductive hormones. Examples of other useful drugs include neurotransmitters such as L-DOPA, antihypertensives or saluretics such as Metolazone from Searle Pharmaceuticals, carbonic anhydrase inhibitors such as Acetazolamide from Lederle Pharmaceuticals, insulin like drugs such as glyburide, a blood glucose lowering drug of the sulfonylurea class, synthetic hormones such as Android F from Brown Pharmaceuticals and TESTRED® (methyltestosterone) from ICN Pharmaceuticals. Representative anti-proliferative agents include, angiogenesis inhibitors, anti-VEGF compounds; receptor tyrosine kinase (RTK) inhibitors such as sunitinib (SUTENT®); tyrosine kinase inhibitors such as sorafenib (NEXAVAR®), erlotinib (TARCEVA®), pazopanib, axitinib, and lapatinib; transforming growth factor-α or transforming growth factor-β inhibitors. In one embodiment, the active agent is sunitinib.

In some embodiments, the agent is an agent for treatment or prevention of a retinal disease, such as a degenerative retinal disease. Exemplary drug types include antioxidant molecules that scavenge and prevent oxidative and nitrosative damage, anti-infectives, corticosteroids, analgesics, nutraceuticals. In a preferred embodiment the agent is a small molecule drug for treating macular degeneration. Exemplary drugs include xanthophylls (Lutein), verteporfin (Visudyne), natamycin (Natacyn), sulfacetamide ophthalmic (Bleph-10), pegaptanib (Macugen), cephalosporin (ceftriaxone), and corticotropin.

### 2. Diagnostic Agents

In some embodiments the formulations include diagnostic agents. These agents can also be used prophylactically. Examples of diagnostic agents include paramagnetic molecules, fluorescent compounds, magnetic molecules, and radionuclides, x-ray imaging agents, and contrast media. Examples of other suitable contrast agents include gases or gas emitting compounds, which are radioopaque.

Exemplary diagnostic agents include dyes such as fluorescent dyes and near infra-red dyes, SPECT imaging agents, PET imaging agents and radioisotopes.

### III. Methods and Preparations of Improved Hypotonic Gel-Forming Compositions

Hypotonic gel-forming compositions including low molecular weight PEG can be prepared as liquids for administration onto the mucosal and/or epithelial surfaces. The formulations are particularly suited for enhanced delivery of therapeutic, diagnostic, prophylactic, or other agents to the eye.

Drug solubilization provides potential advantages that include enhanced physical stability upon storage, increased drug penetration into the body, and a more reproducible drug dose when administered to a patient. epithelial tissues or epithelial surfaces.

Water insoluble drugs are especially challenging to deliver to mucosal surface, such as that of the eye, etc. due to lack of absorption. The formulations are particularly suited for delivering therapeutic agents that are poorly water-soluble. Water-soluble drugs are also challenging to deliver to a mucosal surface in a sustained fashion. Thus, the gelling material can also be used as a vehicle to improve the mucosal delivery of water-soluble drugs by, for example, providing more sustained drug absorption which can reduce side effects and provide more prolonged efficacy.

Further, mucosal surfaces are rapidly cleared and renewed as a normal defense mechanism from infections and foreign particulates. Improving drug solubilization can improve mucosal penetration, and improving distribution, penetration, and retention of hydrophobic drugs is a promising strategy for improving therapeutic effects. Methods solubilization of various drugs and drug complexes with low water solubility into materials that also have thermosensitive gelling properties are described in U.S. publication Nos. 2021/0196837, and 2021/0177751.

In some embodiments, the formulations can be prepared as liquids for administration onto the surface of the eye. The gel forming liquid or polymer solubilizes insoluble drugs by forming micelles. Powder can be made by freeze-drying and reconstituted at the time of use.

The formulations may also include pharmaceutically acceptable diluents, preservatives, solubilizers, stabilizer, emulsifiers, adjuvants and/or carriers. Stabilizers such as SPAN® 20 (sorbitan laurate, CAS Number 1338-39-2) may facilitate dissolution and prevent re-aggregation. Other exemplary stabilizers include polysorbates or TWEENS®, e.g., poly-sorbate 20, polysorbate 60, polysorbate 65 and polysorbate 80, and polyglycerol esters (PGE), polyoxyethylene alkyl ethers, poloxyl stearates, fatty acids (e.g., oleic acid) and propylene glycol monostearate (PGMS). In some cases, the composition includes one or more stabilizers.

Dosage formulations will typically be prepared as single or multiple liquid or dry dosage units in an appropriate applicator, for example, an eye drop dispenser. A person of ordinary skill in the art will be aware of many options for drug storage and application, such as dual chambered devices that may be used to keep various components separate during storage. Multiple dosage units will typically include a barrel loaded with powder, and a plunger having dosage increments thereon. These will typically be sterilized and packaged in sealed, sterile packaging for storage and distribution. See also Remington: The Science and Practice of Pharmacy, 22nd Edition.

Dosage unit administrators may be designed to fit the anatomic location to which drug is to be delivered, such as intraocular administration by one or more eye drops. In an exemplary embodiment, the formulation is a solution having a total volume of between about 0.01 ml and about 2.5 ml, inclusive, for extrusion of drops each of between about 10 μl and about 200 μl, inclusive, e.g., 50 μl/drop).

### IV. Methods of Using Improved Hypotonic Gel-Forming Compositions

The hypotonic gel-forming compositions can, in principle, be applied to any water-absorbent surface, including the ocular surface, as well as other mucosal tissues, to form a gel. Preferably, the formulations are applied as a liquid to a mucosal coating on the epithelial surface (e.g., ocular surface) of a subject in need of a therapeutic, prophylactic, diagnostic, or nutritional effect. The gel-forming composition can be applied to the epithelial surface (e.g., ocular surface) in any number of ways known to the skilled artisan as long as the hypotonic solution, or reagents forming the hypotonic solution, contacts the surface. Suitable epithelial surfaces include ocular surfaces, oral surfaces, pharyngeal surfaces, esophageal surfaces, pulmonary surfaces, aural surfaces, nasal surfaces, buccal surfaces, lingual surfaces, vaginal surfaces, cervical surfaces, genitourinary surfaces, alimentary surfaces, anorectal surfaces, and/or skin surfaces.

By applying the gel-forming compositions as a hypotonic formulation, water is absorbed into the epithelial tissue. Water absorption provides for concentration of the gel-forming polymer at the surface, resulting in uniform gel formation at the surface of the eye. The gel can act as a barrier, reservoir, or combination thereof. Agents or excipients in the gel-forming composition can become entrapped in the gel and can be released at or into the surface of the eye beneath the gel.

Exemplary epithelial surfaces onto which the compositions can be applied include ocular surfaces, as well as oral surfaces, pharyngeal surfaces, esophageal surfaces, pulmonary surfaces, aural surfaces, nasal surfaces, buccal surfaces, lingual surfaces, vaginal surfaces, cervical surfaces, genitourinary surfaces, alimentary surfaces, anorectal surfaces, and/or skin surfaces.

In some examples, the hypotonic gel-forming compositions retain an effective concentration of one or more active agents at or near the site of application for an extended period of time, for example, more than 20 seconds, more than 30 seconds, 40 seconds, 50 seconds, 60 seconds, 70 seconds, 80 seconds, 90 seconds, 100 seconds or more than 100 seconds, for example, 2 minutes, 3, 4, 5, 6, 7, 8, 9, or 10 minutes, up to one hour or more than one hour, for example, 2 hours, more than 2 hours, more than 1 day, more than 2 days, more than 3 days, more than 4 days, more than 5 days, more than 6 days, or more than a week. In some embodiments, the prolonged intraocular residence time of one or more active agents is around or inside one of more cell types in the eye at or near the site of application. In one embodiment, the prolonged intraocular residence time of one or more active agents is about one week.

In some cases, the hypotonic gel-forming compositions increase the concentration of one or more active agents at or near the site of application by 2-fold, 3-fold, 4-fold, 5-fold, 6-fold, 7-fold, 8-fold, 9-fold, 10-fold, 20-fold, 30-fold, 50-fold, 100 fold, or more than 100-fold compared to active agents delivered without gel-forming vehicles, for example, in saline solution.

### A. Methods of Administering

When the hypotonic gel-forming compositions are applied to the eye, the mucosal sites with increased concentration of active agents include one or more of cornea, aqueous humor, sclera, conjunctiva, iris, lens, retina, and retinal pigment epithelium.

The methods can be used to treat or prevent one or more disease or disorders, or to treat or prevent one or more symptoms of one or more diseases or disorders in a subject in need thereof. In some embodiments, the methods deliver an effective amount of an agent to achieve a desired physiological goal or change in the subject. Exemplary physiological changes include variations of the amounts of one or more biomarker in the subject, for example, in the eye of the subject. The change(s) and/or desired outcome of treatment can be monitored to assess the efficacy of treatment, and to determine the amount and extent of treatment required at any given point.

In some embodiments, the improved hypotonic gel-forming compositions deliver active agents (e.g., acriflavine and sunitinib malate) to retina and/or choroid in an amount effective to reduce retinal and/or choroidal neovascularization by 10%, 20%, 30%, 40%, 50%, or more than 50% compared to active agents delivered without gel-forming vehicles, for example, in saline solution.

In other examples, the hypotonic gel-forming compositions deliver active neuroprotective agents (e.g., sunitinib malate) to the retina in an amount effective to increase the survival of retinal ganglion cells following optic nerve injury, and/or to increase the expression of y-synuclein and/or BIII tubulin in retinal ganglion cells following optic nerve injury by 2-fold, 3-fold, 4-fold, 5-fold, or more than 5-fold compared to active agents delivered without gel-forming vehicles, for example, in saline solution.

In further examples, the hypotonic gel-forming compositions deliver active agents (e.g., brinzolamide) to the eye in an amount effective to lower intraocular pressure (IOP) by 10%, 20%, 30%, 40%, 50%, or more than 50% compared to those delivered without gel-forming vehicles, for example, in saline solution within less than 2 hours, 4 hours, 6 hours, 8 hours, 10 hours, 12 hours, or 24 hours.

In other examples, the hypotonic gel-forming compositions deliver active agents (e.g., Cyclosporine A) to the eye in an amount effective to increase tear production by 10%, 20%, 30%, 40%, 50%, or more than 50% compared to those delivered without gel-forming vehicles, for example, in saline solution within less than 2 hours, 4 hours, 6 hours, 8 hours, 10 hours, 12 hours, or 24 hours.

In some embodiments, formulations of hypotonic gel-forming compositions including therapeutic, prophylactic, nutraceutical or diagnostic agents are administered as an eye drop into the eye of the subject.

In some embodiments, the administration to the eye is repeated once or more as part of a treatment regimen, for example, to provide a sufficient concentration of agent(s) at or in the eye. Therefore, in some embodiments, the administration to the eye is repeated at a time selected from hourly, daily, every other day, every three days, every four days, every five days, every six days, weekly, every two weeks, or less often.

### B. Diseases to be Treated

In some embodiments, the hypotonic gel-forming compositions are administered to one or more mucosal epithelial surfaces for delivery of one or more active agents across the epithelium for treatment or prevention of one or more diseases or disorders. Typically, the hypotonic gel-forming compositions including one or more active agents are administered to the surface of the eye of a subject having a disease or disorder to form a hydrogel film across the surface of the eye for the delivery of the one or more active agents to the eye for the treatment of the disease or disorder.

In some embodiments, the methods treat or prevent a disease or disorder including glaucoma, dry eye syndrome (DES), macular degeneration, diabetic retinopathy, scleroderma, and cancer. A preferred method treats or prevents macular degeneration in the eye of a subject.

### 1. Macular Degeneration

In some embodiments, the hypotonic gel-forming compositions are administered to the surface of the eye of a subject for the treatment of macular degeneration in the eye of the subject.

Age-related macular degeneration (AMD) is a common condition that affects the middle part of the vision. It usually first affects people in their 50 s and 60 s, or older. There are two basic types of Macular Degeneration: “dry” and “wet.” Approximately 85% to 90% of the cases of Macular Degeneration are the “dry” (atrophic) type, while 10-15% are the “wet” (exudative) type.

In some embodiments, the hypotonic gel-forming compositions are administered to the surface of the eye of a subject for the treatment of dry macular degeneration in the eye of the subject. Dry macular degeneration is a common eye disorder among people over 50. It causes blurred or reduced central vision, due to thinning of the macula. The macula is the part of the retina responsible for clear vision in the direct line of sight.

The treatment for early dry AMD is generally nutritional therapy, with a healthy diet high in antioxidants to support the cells of the macula. If AMD is further advanced but still dry, supplements are prescribed, to add higher quantities of certain vitamins and minerals which may increase healthy pigments and support cell structure. Therefore, in some embodiments, the hypotonic gel-forming compositions including one or more nutraceutical agents are administered to the surface of the eye of a subject for the treatment of dry macular degeneration in the eye of the subject.

In other embodiments, the hypotonic gel-forming compositions are administered to the surface of the eye of a subject for the treatment of wet macular degeneration in the eye of the subject. In the “wet” type of macular degeneration, abnormal blood vessels (known as choroidal neovascularization or CNV) grow under the retina and macula. These new blood vessels may then bleed and leak fluid, causing the macula to bulge or lift up from its normally flat position, thus distorting or destroying central vision. Under these circumstances, vision loss may be rapid and severe. VEGF is an acronym for. Currently, the most common and effective clinical treatment for wet Age-related Macular Degeneration is anti-vascular endothelial growth factor (anti-VEGF therapy) using an inhibitor of VEGF (anti-VEGF). VEGF is a molecule which supports the growth of new blood vessels. In the case of wet AMD, VEGF promotes the growth of new, weak blood vessels in the choroid layer behind the retina, and those vessels leak blood, lipids, and serum into the retinal layers. The leakage (hemorrhaging) causes scarring in the retina and kills macular cells, including photoreceptor rods and cones. Therefore, in some embodiments, the hypotonic gel-forming compositions including one or more anti-VEGF agents are administered to the surface of the eye of a subject for the treatment of wet macular degeneration in the eye of the subject.

In other embodiments, the hypotonic gel-forming compositions are administered to the surface of the eye of a subject for the treatment of Stargardt disease. Stargardt disease is a form of macular degeneration found in young people, caused by a recessive gene.

### 2. Dry Eye Syndrome (DES)

In some embodiments, the hypotonic gel-forming compositions are administered to the surface of the eye of a subject for the treatment of dry eye syndrome (DES) in the eye of the subject.

Dry eye syndrome (DES), also known as keratoconjunctivitis sicca (KCS), or dry eye disease, is a common condition that occurs when the eyes do not make enough tears, or the tears evaporate too quickly. Other associated symptoms include irritation, redness, discharge, and easily fatigued eyes. Blurred vision may also occur. Symptoms range from mild and occasional to severe and continuous. Scarring of the cornea may occur in untreated cases. The cornea includes the clear outer dome of the eye that allows light to enter and become focused through the lens onto the retina. The cornea is an avascular tissue and receives most of its nutrients from the tears, the air, and fluid inside the eye.

Dry eye occurs when either the eye does not produce enough tears or when the tears evaporate too quickly. This can result from contact lens use, meibomian gland dysfunction, pregnancy, Sjögren syndrome, vitamin A deficiency, omega-3 fatty acid deficiency, LASIK surgery, and certain medications such as antihistamines, some blood pressure medication, hormone replacement therapy, and antidepressants. Chronic conjunctivitis such as from tobacco smoke exposure or infection may also lead to the condition. Diagnosis is mostly based on the symptoms, though a number of other tests may be used.

Treatment options include drugs to reduce eyelid inflammation (e.g., antibiotics), drugs to control cornea inflammation (e.g., immune-suppressing medication cyclosporine (Restasis) or corticosteroids), or tear-stimulating drugs (e.g., cholinergics (pilocarpine, cevimeline)). Therefore, in some embodiments, the hypotonic gel-forming compositions including one or more antibiotic, anti-inflammatory or cholinergenic agents are administered to the surface of the eye of a subject for the treatment of DES in the eye of the subject.

The present invention will be further understood by reference to the following non-limiting examples.

### EXAMPLES

### Example 1: Addition of PEG to Gel-Forming Eye Drop Formulation Increases Tear Break-Up Time

### Methods and Materials

### Materials

Endotoxin-free ultra-pure water, poly(ethylene glycol)-block-poly(propylene glycol)-block-poly(ethylene glycol) (F127), carboxymethylcellulose sodium (CMC) United States Pharmacopeia (USP) Reference Standard, fluorescein sodium, polyethylene glycol 400 (PEG400) USP Reference Standard, polyethylene glycol 300 (PEG300) USP Reference Standard (PEG400), propylene glycol (PG), and polysorbate 80 (PS80) were purchased from Sigma Aldrich. Normal saline (0.9% NaCl), Systane® ULTRA eye drops lubricant high performance, and GenTeal® Tears Lubricant eye ointment were purchased from Alcon.

### Rheology Samples

For sample preparation for rheology studies, 16% (w/v) F127 and other additives (0.025-1% PEG300, PEG400, and glycerin; 0.4% PG, 0.5% PS80, 1% CMC) were mixed together before reconstitution and mixing with purified water. Note that 16% F127 was used for in vitro studies to allow for characterization in the gel state, whereas in vivo studies employed the hypotonic 12% gel-forming formulation.

### Tear Break Up Time (TBUT) Samples

For the tear break up time (TBUT) studies, 5 mg/mL fluorescein sodium powder was added to the various formulations (saline, GenTeal® Tears, Systane® ULTRA, 12% F127, with 0.4% PEG400) prior to mixing. All formulations were freshly prepared for each experiment by mixing for 3 days on a Benchmark Scientific waving rocker with 30° tilt angle and 30 rpm speed at 4° C. All formulations remained wrapped to avoid light exposure and storage at 4° C. when not in use.

### Eyedrop Bottle Force Measurement

Formulations were prepared in 7 mL volume and transferred to 10 mL dropper bottles (Steri-Dropper). A FG-3005 Digital Force Gauge system with 50N Capacity (Nidac) was used to measure the bottle squeezing force. The force gauge was fixed to a syringe pump (NE300), which provided the constant forward displacement to squeeze the bottles. The force probe was placed perpendicularly 1.5 cm from the bottom of the inverted bottle, which was loosely held by a metal clamp. The force probe was moved at a constant rate of 1 mm/s to squeeze the bottle. The force was recorded when the first drop of liquid was dispensed from the bottle tip. For the measurements conducted at 33° C. and 37° C., the whole system was placed in a heated oven and allowed to equilibrate for 15 min prior to performing the measurements.

### Rheology Studies

An Anton Paar cone and plate rheometer (model MCR 302) with PP25 probe were used to measure the max tack force and the adhesion. The probe was placed in contact with the sample, and then moved at a controlled rate vertically away from the sample to measure the normal force. The peak force on the curve is considered the max tack force, while the adhesion is the area under the force curve. Samples (200 μL) were transferred to the rheometer sample holder using a Drummond Wiretrol 200 μL wire plunger pipette. The samples were equilibrated between the probe and sample holder with a 1 mm gap for 60 s at the specified temperature. A total of 100 points were measured at 60 s intervals with ramp linear mode (initial was set as −0.005 mm/s and the final was set as −0.5 mm/s). If not specified, n=3-6 per each sample group were measured and each sample were prepared individually. The final data was exported from the Antron Parr RheoCompass. The max tack force and the adhesion were calculated with GraphPad Prism 9. For the temperature ramp viscosity experiment, the loading gap was set to 0.3 mm. A total of 26 points with 0.25 min temperature change (ramp linear mode) were selected with additional parameters gamma=1 (1/s) and temperature ramp from 15-40° C. (n=3). The raw data with temperature (° C.) and viscosity (mPa*s) measurements were exported from the Anton Parr RheoCompass software and analyzed/plotted with GraphPad Prism 9. Statistical analyses of two groups were conducted using two-tailed Student's t-test. For the comparison of multiple groups, one-way ANOVA with Dunnett's multiple comparison test was used. Statistical analysis was done using GraphPad Prism 9.

### Tear Break Up Time (TBUT)

Pluronic F127 was formulated hypotonically at 12% w/v containing 0.4% PEG400. Several commercial eye drops, including normal saline and lubricating eye drops from the SYSTANE and GENTEAL product lines, and 12% F127 with 0.4% PEG400, had 0.5 mg/mL fluorescein sodium added for visualization purposes. Tear break up time (TBUT) was performed in healthy New Zealand White rabbits (n=4 different eyes per group with two independent measurements made by masked observers). Eye drops containing fluorescein were dosed at 50 μL volume to conscious rabbits wrapped gently in a towel. After administration, the lid was closed manually twice to distribute the tear film and agent. The eye was then gently held open, and the TBUT was measured and recorded. The TBUT was scored by two individuals agreeing and confirming the time in a masked manner without knowing the formulation identity. As shown, the hypotonic 12% F127 with 0.4% PEG400 demonstrated a significant increase in TBUT compared to top performing commercial lubricating eye drop formulations, *p<0.05. For the comparison of multiple groups, one-way ANOVA with Dunnett's multiple comparison test was used. Statistical analysis was done using GraphPad Prism 9.

### Results

### Effect of Glycerin on Max Tack Force

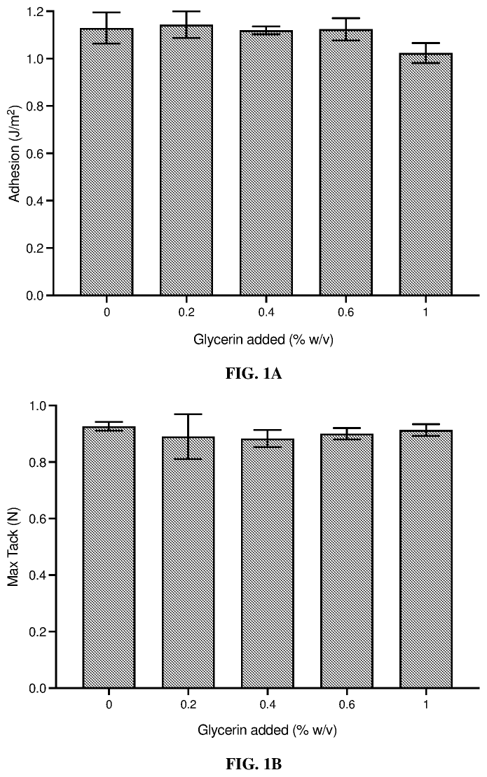

Pluronic F127 was formulated at 16% w/v to remain above the critical gel concentration (CGC) to facilitate in vitro gelation at 37° C. and evaluation of gel rheological properties. Various amounts of glycerin (0.2-1%, 0% represents F127 with no additives) was added to characterize the effect on (FIG. 1A) adhesion and (FIG. 1B) max tack force of the F127 gels (n=3-6). The addition of glycerin had no measurable effect.

### Effect of PEG300 on Max Tack Force

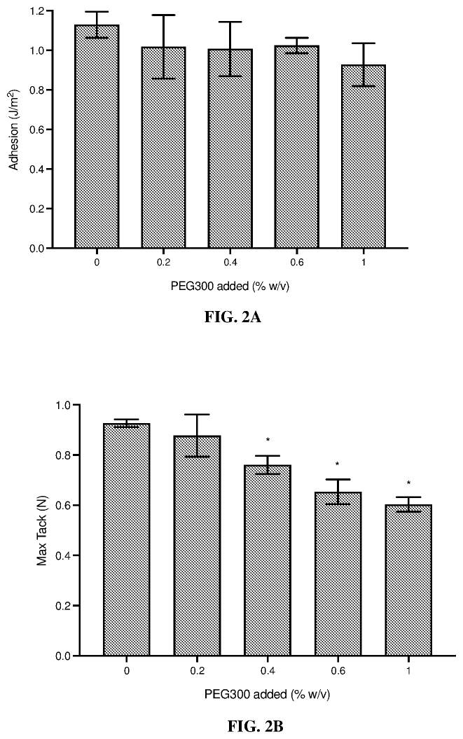

Pluronic F127 was formulated at 16% w/v to remain above the critical gel concentration (CGC) to facilitate in vitro gelation at 37° C. and evaluation of gel rheological properties. Various amounts of PEG300 (0.2-1%, 0% represents F127 with no additives) was added to characterize the effect on (FIG. 2A) adhesion and (FIG. 2B) max tack force of the F127 gels (n=3-6). The max tack force was significantly reduced with the addition of 0.4-1% PEG300.

### Effect of PEG400 on Max Tack Force

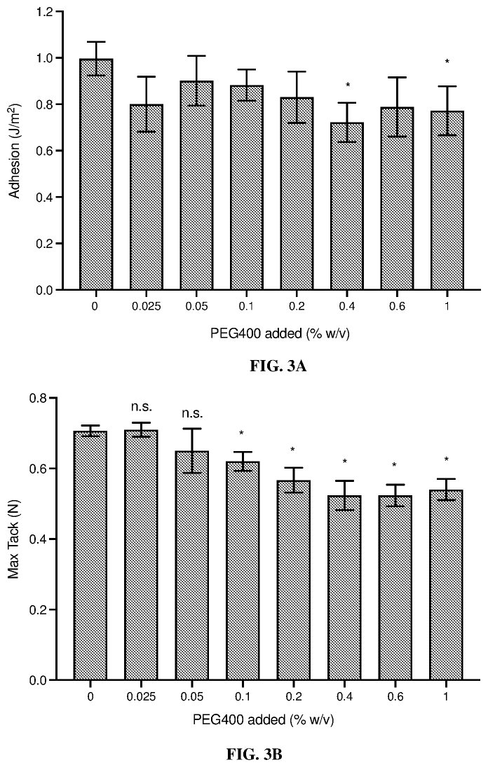

Pluronic F127 was formulated at 16% w/v to remain above the critical gel concentration (CGC) to facilitate in vitro gelation at 37° C. and evaluation of gel rheological properties. Various amounts of PEG400 (0.025-0.1%, 0% represents F127 with no additives) was added to characterize the effect on (FIG. 3A) adhesion and (FIG. 3B) max tack force of the F127 gels (n=3-6). The max tack force was significantly reduced with the addition of 0.1%-1% PEG400.

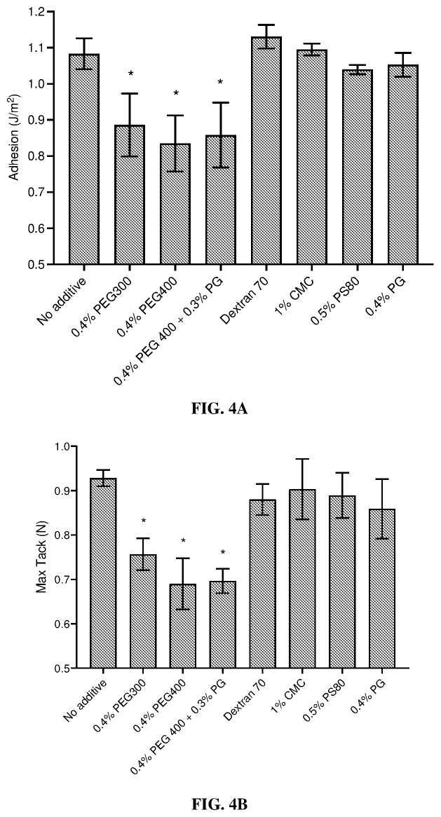

Effect of PEG300 and PEG400 on Max Tack Force Pluronic F127 was formulated at 16% w/v to remain above the critical gel concentration (CGC) to facilitate in vitro gelation at 37° C. and evaluation of gel rheological properties. Various lubricating and demulcent materials were added to characterize the effect on (FIG. 4A) adhesion and (FIG. 4B) max tack force of the F127 gels (n=3-6). The addition of PEG300 and PEG400 reduced the max tack, with the most pronounced effect observed with PEG400.

### Changes in Viscosity

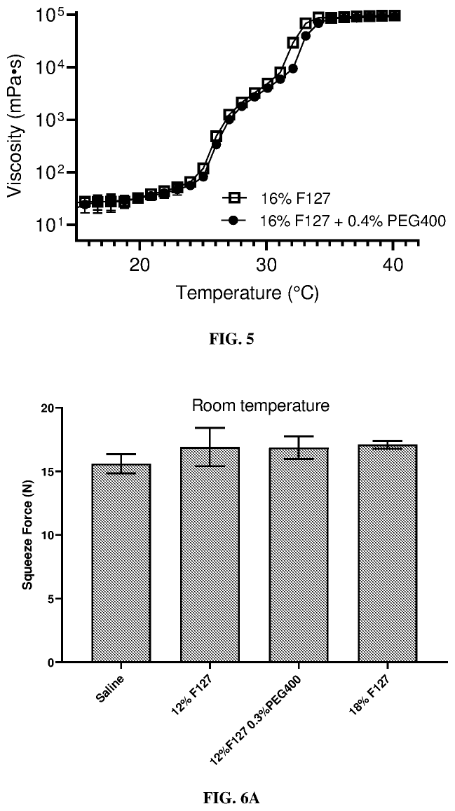

Pluronic F127 was formulated at 16% w/v to remain above the critical gel concentration (CGC) to facilitate in vitro gelation and evaluation of gel rheological properties. A constant stress temperature ramp was conducted for over 15-40° C. to evaluate the change in viscosity with temperature with and without the addition of 0.4% PEG400. The viscosity profile as a function of temperature was not significantly affected by the presence of the 0.4% PEG400 (FIG. 5). Thus, reducing the max tack force with the addition of PEG400 does not affect the thermoreversible gelling behavior of F127.

### Effects on Squeeze Force

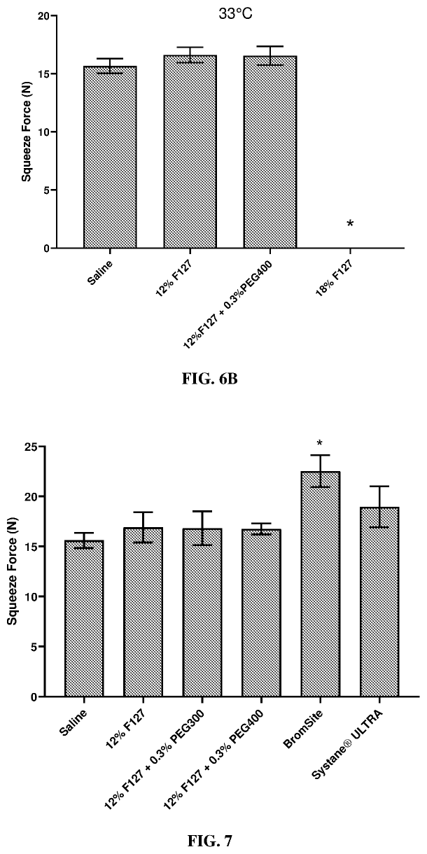

Saline, 12% F127, 12% F127 with 0.3% PEG400, and 18% F127 were added to standard eye dropper bottles to measure the squeeze force required to expel the first droplet at (FIG. 6A) room temperature or (FIG. 6B) 33° C. to mimic storage in a clothing pocket. *At 33° C., the 18% F127 formed a gel and was extruded as a band of gel rather than a droplet. In contrast, formulation at 12% F127 (below the CGC), prevents premature gelation

Saline, 12% F127, 12% F127 with 0.3% PEG300, 12% F127 with 0.3% PEG400, BromSite, and Systane ULTRA were added to standard eye dropper bottles to measure the squeeze force required to expel the first droplet at room temperature (FIG. 7). Only BromSite required a significantly higher squeeze force than saline (p<0.05), whereas F127 formulations were indistinguishable from saline.

Effects of PEG400 on Tear Break-Up time

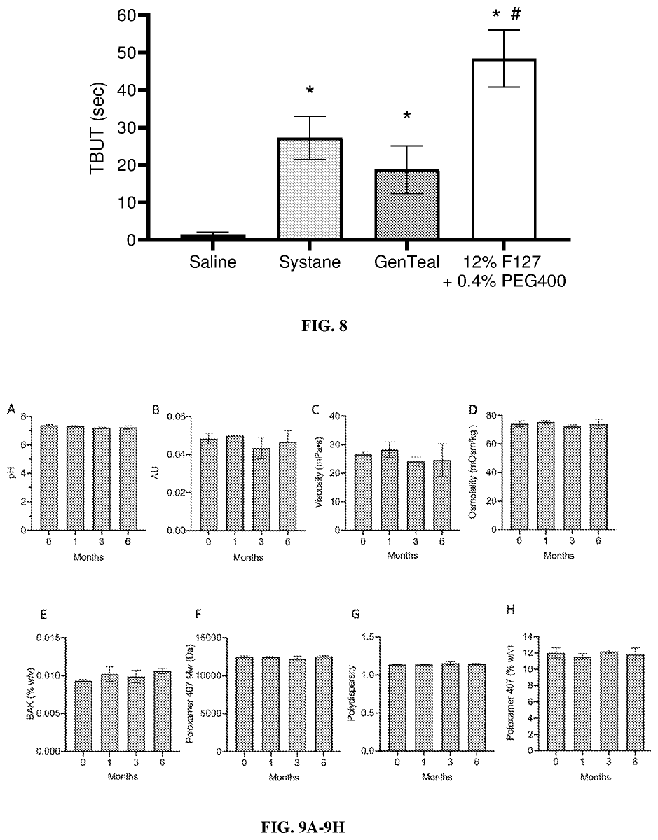

Pluronic F127 was formulated hypotonically at 12% w/v, a concentration below the critical gel concentration (CGC) that was shown to provide a thin, uniform gel layer in vivo (though does not form a gel in vitro). Several commercial eye drops, including normal saline and lubricating eye drops from the Systane and GenTeal product lines, and 12% F127 with 0.4% PEG400, had 0.5 mg/mL fluorescein sodium added for visualization purposes. Tear break up time (TBUT) was performed in healthy New Zealand White rabbits (n=4 different eyes per group with two independent measurements made by masked observers). As shown in FIG. 8, the hypotonic 12% F127 with 0.4% PEG400 demonstrated a significant increase in TBUT compared to top performing commercial lubricating eye drop formulations.

### Example 2: Stability Study and Sterility Tests

### Materials & Methods

OcuGel containing 12% F127, 0.4% PEG400 and 1 mM borate buffer was formulated with or without 0.01% benzalkonium chloride (“BAK”) at either 72 mOsm or 150 mOsm. For pH measurement, 300 μL of each sample was transferred to 1.5 mL EPPENDORF® and measured with the Mettler Toledo EL20 pH meter with micro electrode.

For absorbance measurement, 200 μL of each sample was transferred to the Nunc™ MicroWell™ 96-Well Microplates and the absorbance at λ=490 nm and was measured with the absorbance plate reader (Synergy MTX reader). A Modular Compact Rheometer (MCR302) with PP25 parallel plate measuring system (Anton Parr) was used to measure the sample viscosity (mPa*s). A Drummond WIRETROL® II 200 μL with wire plunger was used to disperse 200 μL of the samples on the 37° C. mount. The location of PP25 probe were set at 0.5 mm with 1 minute temperature equilibration between the formulation and the probe. Ramp linear mode with initial 1 (1/s) and final 100 (1/s) were set with a total of 10 measurement point, each with 10 seconds measurement periods. Viscosity values (mPa*s) at 100 (1/s) were then reported for each timepoint for stability comparison.

For osmolality measurement, the Vapro osmometer system (ELITechGroup) were used to measure the sample osmolality.

The BAK concentration, when included in the formulation, was measured with high-performance liquid chromatography (HPLC, Prominence LC2030, Shimadzu) and with LUNA® 5 μm C18 100 Å, 00G-422-E0 column (Phenomenex). Acetonitrile and water were used as a mobile phase at a ratio of 75:25. Samples were eluted isocratically at a flow rate of 1 mL/min through a C18-reversed phase column at 40° C. UV absorbance was monitored at 205 nm and the area under the curve (AUC) were used to calculate the BAK concentration.

To measure the F127 molecular weight (Mw), 100 μL of the formulation was frozen at −80° C. and lyophilized. DMF with 0.1% LiBr solution (1.2 mL) was added to the sample and vortexed to dissolve. A gel permeation chromatography system (1260 Infinity II series, Agilent Technologies) with organic column (Agilent, 10 æm MIXED-B Columns) was used to separate and detect the F127 with 1 mL/min flow rate and pressure set near 33 bar. The Mw and polydispersity (PD) values were calculated using Alginate software (Version 1.4).

OcuGel with and without preservative was formulated in small batches, sterile filtered, and aliquoted into sterilized eye dropper bottles in a biosafety cabinet. The dropper bottles were capped and wrapped with heat shrink wrap sealer and stored at room temperature in a secured cabinet. Ten bottles per time point (0, 3 months) were stored at room temperature in a dark cabinet. At each time point, the 10 bottles were shipped to Pace Analytical® Life Sciences for pooling and sterility testing according to the <USP 71> guidelines.

### Safety Studies

Acute: OcuGel (12% Poloxamer 407, 0.4% PEG400, 1 mM borate buffer, 0.01% BAK, pH 7.4) was formulated in small batches, sterile filtered, and aliquoted into sterilized eye dropper bottles in a biosafety cabinet.

All animals were handled and treated following the Association for Research in Vision and Ophthalmology Statement for the Use of Animals in Ophthalmic and Vision Research. Equal numbers of male and female New Zealand White rabbits were used in the topical acute toxicity study.

For the acute dosing study, n=4 rabbits were dosed with 50 μL of OcuGel in the right eye, and 50 μL of SYSTANE® ULTRA in the left eye each hour for 8 hours. After the last dose, a board-certified ophthalmologist anesthetized the rabbits and performed ocular evaluations.

Cornea neovascularization was graded 0-2 scale (0 indicates normal function, 2 indicates severely injured or inflamed). The pupillary light reflex, conjunctival hyperemia, and conjunctival discharge were graded in a 0-3 scale, where 0 indicates normal function and 3 as severe injured or inflamed. The conjunctival swelling, cornea opacity (severity), corneal (area), anterior chamber cells, iris involvement, anterior vitreous cells, eyelid discharge, eyelid swelling, eyelid vascularity, meibomian gland function, and fluorescein staining were graded in a 0-4 scale (0 indicates normal function and 4 as severely injured or inflamed). After the examination, the rabbits were closely monitored in the home cage until they fully awakened.

Chronic: OcuGel (12% POLOXAMER 407, 0.4% PEG400, 1 mM borate buffer, 0.01% BAK, pH 7.4) was formulated in small batches, sterile filtered, and aliquoted into sterilized eye dropper bottles in a biosafety cabinet. All animals were handled and treated following the Association for Research in Vision and Ophthalmology Statement for the Use of Animals in Ophthalmic and Vision Research. Equal numbers of male and female New Zealand White rabbits were used in the topical acute toxicity study. For the acute dosing study, n=4 rabbits were dosed with 50 μL of OcuGel in the right eye, and 50 μL of SYSTANE® ULTRA in the left eye Three times per day for 14 days. After the last dose on days 7 and 14, a board-certified ophthalmologist anesthetized the rabbits and performed ocular evaluations. Cornea neovascularization was graded 0-2 scale (0 indicates normal function, 2 indicates severely injured or inflamed). The pupillary light reflex, conjunctival hyperemia, and conjunctival discharge were graded in a 0-3 scale, where 0 indicates normal function and 3 as severe injured or inflamed. The conjunctival swelling, cornea opacity (severity), corneal (area), anterior chamber cells, iris involvement, anterior vitreous cells, eyelid discharge, eyelid swelling, eyelid vascularity, meibomian gland function, and fluorescein staining were graded in a 0-4 scale (0 indicates normal function and 4 as severely injured or inflamed). After the examination, the rabbits were closely monitored in the home cage until they fully awakened.

Tear Break Up Time (TBUT) measurements

OcuGel containing 12% F127, 0.4% PEG400, 1 mM borate buffer, and 0.01% BAK was formulated with or without 0.2% HA (2 MDa) at various osmolalities (100, 150, 200, 250, 300 mOsm/kg) and adjusted to pH 7-7.4. Normal saline and Systane® Hydration PF were used as comparators. TBUT was performed in healthy New Zealand White rabbits (n=3). Various formulations were first dosed topically (50 μL). After 90 minutes, 50 μL of 2% fluorescein disodium at pH 7.4 was dosed on the treated eyes. After administration, the lid was closed manually twice to distribute the tear film and agent. The eye was then gently held open, and the TBUT was measured and recorded. The TBUT was scored by two individuals agreeing and confirming the time in a masked manner without knowing the formulation identity, n=3 independent animals. Data are mean±SD. Statistical analyses conducted by one-way ANOVA with multiple comparison with respect to the OcuGel 72 mOsm/kg group.

### Results

### Room Temperature Stability and Sterility Test

OcuGel (12% Poloxamer 407, 0.4% PEG400, 1 mM borate buffer, 0.01% BAK) was formulated in small batches, sterile filtered, and aliquoted into sterilized eye dropper bottles in a biosafety cabinet. The dropper bottles were capped and wrapped with heat shrink wrap sealer and stored at room temperature in a secured cabinet. Three bottles per time point (0, 1, 3, 6 months) were stored at room temperature in a dark cabinet. For each time point, the samples were characterized for pH, absorbance, viscosity, osmolality, BAK concentration, and polymer molecular weight.

FIGS. 9A-9H are graphs of OcuGel without preservative (n=3) stability at room temperature for up to 3 months. Measurements of (9A) pH, (9B) absorbance (AU), (9C) viscosity (mPa*s, shear rate 100 s−1), (9D) osmolality (mOsm/kg), (9E) BAK concentration (% w/v), and Poloxamer 407 (9F) molecular weight (Mw), (9G) polydispersity and (9H) % w/v in the OcuGel formulation.

FIGS. 10A-10G are graphs of OcuGel (n=3) stability at room temperature for up to 6 months. Measurements of (10A) pH, (10B) absorbance (AU), (10C) viscosity (mPa*s, shear rate 100 s−1), (10D) osmolality (mOsm/kg), and Poloxamer 407 (10E) molecular weight (Mw), (10F) polydispersity and (10G) % w/v in the formulation.

The pH remained within the range of 7.0-7.4 (FIG. 9A, 10A), the absorbance remained in the range of 0.04-0.06 AU (FIG. 9B,10B), the viscosity remained in the range of 20-30 mPa*s (FIG. 9C,10C), the osmolality remained in the range of 70-80 mOsm/kg (FIG. 9D,10D), the BAK concentration remained within 0.009-0.011% (w/v) (FIG. 9E), the Poloxamer 407 molecular weight (Mw) remained within 12,000-13,000 Da (FIG. 9F,10E), the Poloxamer 407 polydispersity (PD) remained within 1.0-1.2 (FIG. 9G,10F), and the Poloxamer 407 concentration remained within 11.5-12.5% (w/v) (FIG. 9H, 10G).

### Sterility

Table 1 shows that OcuGel with BAK preservative met the sterile requirement for <USP 71> (A) 0 and (B) 3 months, and OcuGel without preservative met the sterile requirement for <USP 71> at (C) 3 months. All OcuGel formulations tested passed the sterility test according to USP <71>

### Preclinical Safety Testing

The grading shown for the acute toxicity study (Table 2) and in the chronic study at day 7 (Table 3) and day 14 (Table 4) showed essentially no notable evidence of ocular irritation with either treatment regimen.

### Tear Break Up Time (TBUT)

OcuGel (12% Poloxamer 407, 0.4% PEG400, 1 mM borate buffer, 0.01% BAK) was formulated with and without the addition of 0.2% HA at various osmolalities (72-300 mOsm/kg) and dosed to NZW rabbits. Fluorescein-based TBUT was assessed at 90 min after dosing.

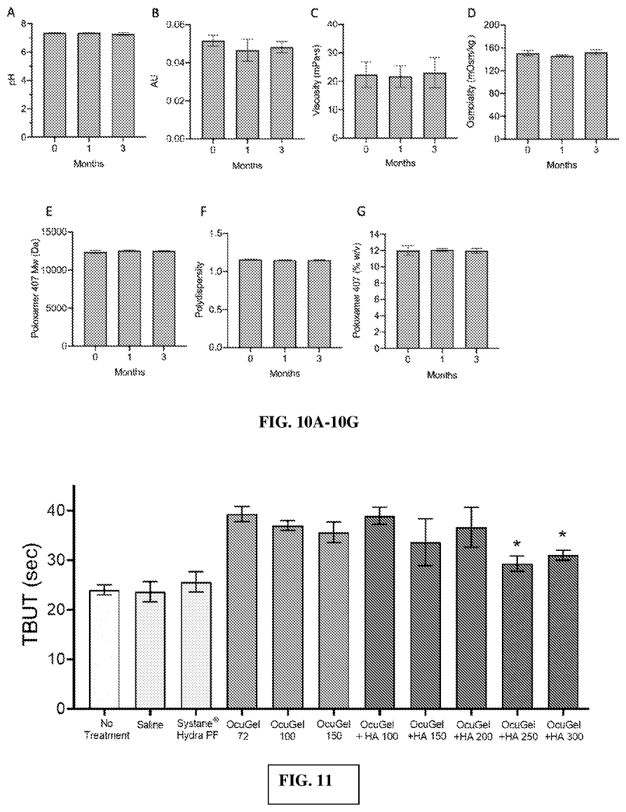

FIG. 11 is a graph of OcuGel (12% Poloxamer 407, 0.4% PEG400, 1 mM borate buffer, 0.01% BAK) with or without the addition of 0.2% HA at various osmolality (72-300 mOsm/kg) dosed to NZW rabbits, measured as Fluorescein-based tear break up time (TBUT) at 90 min after dosing, compared to no treatment, saline, and SYSTANE® HYDRATION PF. *p<0.05 compared to OcuGel at 72 mOsm/kg.

As shown in FIG. 11, TBUT returned to baseline levels of untreated rabbits in the saline and Systane® Hydration PF groups. In contrast, all OcuGel treated animals, except the OcuGel +HA 250 mOsm/kg group, had increased TBUT compared to untreated animals at 90 min. However, the HA formulations with higher osmolality, including 250 and 300 mOsm/kg, had reduced TBUT compared to OcuGel at 72 mOsm. The data supported the long-lasting surface lubrication effect, including when additional lubricating polymers such as HA were added, as well as the importance of the hypotonic formulation for achieving these benefits.

Tables 1-4 demonstrate the sterility and safety of the formulations.

TABLE 1
Results of Sterility Tests
Analysis Performed: LM 246, Sterility by Membrane Filtration
Effective date: 29 Jun. 2021 (testing in compliance with cGMPs)

| Sample ID | Analysis date | Specification | Result |
|---|---|---|---|
| OcuGel with | 17 May 2022 | Meets the | Complies: |
| preservative, 0 | | Requirements | TSB: No Growth |
| month | | (Sterile) | FTM: No Growth |
| OcuGel with | 11 Aug. 2022 | No growth | Complies: |
| preservative, 3 | | | TSB: No Growth |
| months | | | FTM: No Growth |
| OcuGel without | 25 Oct. 2022 | No growth | Complies: |
| preservative, 3 | | | TSB: No Growth |
| months | | | FTM: No Growth |

TABLE 2
Acute Toxicity Study
Ocular grading for the acute (8 doses hourly over 8 hours)
dosing of OcuGel and Systane ® Ultra in NZW rabbits (n = 4 eyes).

| | OcuGel (Right | | | | Systane Ultra ® | | | |
|---|---|---|---|---|---|---|---|---|
| | eyes) | | | | (Left eyes) | | | |
| Exam | 302 | 301 | 304 | 303 | 302 | 301 | 304 | 303 |
| Pupillary Light Reflex | 0 | 0 | 0 | 0 | 0 | 0 | 0 | 0 |
| Conjunctival Hypermia | 0 | 0 | 0 | 0 | 0 | 0 | 0 | 0 |
| Conjunctival Swelling | 0 | 0 | 0 | 0 | 0 | 0 | 0 | 0 |
| Conjunctival Discharge | 0 | 0 | 0 | 0 | 0 | 0 | 0 | 0 |
| Corneal Opacity (severity) | 0 | 0 | 0 | 0 | 0 | 0 | 0 | 0 |
| Corneal Opacity (area) | 0 | 0 | 0 | 0 | 0 | 0 | 0 | 0 |
| Corneal Vascularization | 0 | 0 | 0 | 0 | 0 | 0 | 0 | 0 |
| Aqueous Flare | 0 | 0 | 0 | 0 | 0 | 0 | 0 | 0 |
| Anterior Chamber Cells | 0 | 0 | 0 | 0 | 0 | 0 | 0 | 0 |
| Iris Involvement | 0 | 0 | 0 | 0 | 0 | 0 | 0 | 0 |
| Anterior Vitreous Cells | 0 | 0 | 0 | 0 | 0 | 0 | 0 | 0 |
| Fluorescein Staining (severity) | 0 | 0 | 0 | 0 | 0 | 0 | 0 | 0 |
| Fluorescein Staining (area) | 0 | 0 | 0 | 0 | 0 | 0 | 0 | 0 |
| Eyelid Discharge | 0 | 0 | 0 | 0 | 0 | 0 | 0 | 0 |
| Eyelid Swelling | 0 | 0 | 0 | 0 | 0 | 0 | 0 | 0 |
| Eyelid Vascularity | 0 | 0 | 0 | 0 | 0 | 0 | 0 | 0 |
| Meibomian Gland Function | 0 | 0 | 0 | 0 | 0 | 0 | 0 | 0 |

TABLE 3
Ocular grading for the chronic (three times daily dosing for
14 days) dosing of OcuGel and Systane ® Ultra in
NZW rabbits (n = 4 eyes) after the last dose on day 7.

| | OcuGel | | | | Systane Ultra ® | | | |
|---|---|---|---|---|---|---|---|---|
| Day 7 | (Right eyes) | | | | (Left eyes) | | | |
| Exam | 302 | 301 | 304 | 303 | 302 | 301 | 304 | 303 |
| Pupillary Light Reflex | 0 | 0 | 0 | 0 | 0 | 0 | 0 | 0 |
| Conjunctival Hypermia | 0 | 0 | 0 | 0 | 0 | 0 | 0 | 0 |
| Conjunctival Swelling | 0 | 0 | 0 | 0 | 0 | 0 | 0 | 0 |
| Conjunctival Discharge | 0 | 0 | 0 | 0 | 0 | 0 | 0 | 0 |
| Corneal Opacity (severity) | 0 | 0 | 0 | 0 | 0 | 0 | 0 | 0 |
| Corneal Opacity (area) | 0 | 0 | 0 | 0 | 0 | 0 | 0 | 0 |
| Corneal Vascularization | 0 | 0 | 0 | 0 | 0 | 0 | 0 | 0 |
| Aqueous Flare | 0 | 0 | 0 | 0 | 0 | 0 | 0 | 0 |
| Anterior Chamber Cells | 0 | 0 | 0 | 0 | 0 | 0 | 0 | 0 |
| Iris Involvement | 0 | 0 | 0 | 0 | 0 | 0 | 0 | 0 |
| Anterior Vitreous Cells | 0 | 0 | 0 | 0 | 0 | 0 | 0 | 0 |
| Fluorescein Staining (severity) | 0 | 0 | 0 | 0 | 0 | 0 | 0 | 0 |
| Fluorescein Staining (area) | 0 | 0 | 0 | 0 | 0 | 0 | 0 | 0 |
| Eyelid Discharge | 0 | 0 | 0 | 0 | 0 | 0 | 0 | 0 |
| Eyelid Swelling | 0 | 0 | 0 | 0 | 0 | 0 | 0 | 0 |
| Eyelid Vascularity | 0 | 0 | 0 | 0 | 0 | 0 | 0 | 0 |
| Meibomian Gland Function | 0 | 0 | 0 | 0 | 0 | 0 | 0 | 0 |

TABLE 4
Ocular grading for the chronic (three times daily dosing for
14 days) dosing of OcuGel and Systane ® Ultra in NZW
rabbits (n = 4 eyes) after the last dose on day 14.

| | OcuGel | | | | Systane Ultra ® | | | |
|---|---|---|---|---|---|---|---|---|
| Day 14 | (Right eyes) | | | | (Left eyes) | | | |
| Exam | 302 | 301 | 304 | 303 | 302 | 301 | 304 | 303 |
| Pupillary Light Reflex | 0 | 0 | 0 | 0 | 0 | 0 | 0 | 0 |
| Conjunctival Hypermia | 0 | 0 | 0 | 0 | 0 | 0 | 0 | 0 |
| Conjunctival Swelling | 0 | 0 | 0 | 0 | 0 | 0 | 0 | 0 |
| Conjunctival Discharge | 0 | 0 | 0 | 0 | 0 | 0 | 0 | 0 |
| Corneal Opacity (severity) | 0 | 0 | 0 | 0 | 0 | 0 | 0 | 0 |
| Corneal Opacity (area) | 0 | 0 | 0 | 0 | 0 | 0 | 0 | 0 |
| Corneal Vascularization | 0 | 0 | 0 | 0 | 0 | 0 | 0 | 0 |
| Aqueous Flare | 0 | 0 | 0 | 0 | 0 | 0 | 0 | 0 |
| Anterior Chamber Cells | 0 | 0 | 0 | 0 | 0 | 0 | 0 | 0 |
| Iris Involvement | 0 | 0 | 0 | 0 | 0 | 0 | 0 | 0 |
| Anterior Vitreous Cells | 0 | 0 | 0 | 0 | 0 | 0 | 0 | 0 |
| Fluorescein Staining (severity) | 0 | 0 | 0 | 0 | 0 | 0 | 0 | 0 |
| Fluorescein Staining (area) | 0 | 0 | 0 | 0 | 0 | 0 | 0 | 0 |
| Eyelid Discharge | 0 | 0 | 0 | 0 | 0 | 0 | 0 | 0 |
| Eyelid Swelling | 0 | 0 | 0 | 0 | 0 | 0 | 0 | 0 |
| Eyelid Vascularity | 0 | 0 | 0 | 0 | 0 | 0 | 0 | 0 |
| Meibomian Gland Function | 0 | 0 | 0 | 0 | 0 | 0 | 0 | 0 |

Those skilled in the art will recognize, or be able to ascertain using no more than routine experimentation, equivalents to the specific embodiments of the invention described herein. Such equivalents are intended to be encompassed by the following claims.

1. A formulation for delivery of a therapeutic, prophylactic, diagnostic or nutraceutical agent to the eye comprising
a gel-forming polymer for application to a mucosal or epithelial surface formulated so that it is at a concentration below the critical gel concentration (CGC) of the polymer under isotonic conditions and a temperature between room temperature and body temperature (about 25 to about 37° C.),
a low-molecular weight polyethylene glycol (PEG) in an amount between about 0.1% and about 1.0% inclusive, weight/volume (w/v) of the total, and
excipients to form a pharmaceutically acceptable hypotonic formulation of the polymer suitable for delivery to the mucosal or epithelial surface of an individual in need thereof,
optionally including a therapeutic, prophylactic, nutraceutical, or diagnostic agent.

2. The formulation of claim 1, wherein the gel-forming polymer is a thermosensitive gel-forming polymer.

3. The formulation of claim 2, wherein the thermosensitive gel-forming polymer has a lower critical solution temperature that is below 30° C., preferably below 21° C.

4. The formulation of claim 1, wherein the gel-forming polymer is between greater than 10% and less than 18% in an aqueous excipient.

5. The formulation of claim 1, wherein the gel-forming polymer is a poloxamer.

6. The formulation of claim 5, wherein the gel forming polymer is poly(ethylene glycol)-block-poly(propylene glycol)-block-poly(ethylene glycol) (Pluronic F127).

7. The formulation of claim 1, wherein the gel-forming polymer is between 10% and 16% F127.

8. The formulation of claim 1, wherein the PEG is one or more selected from the group consisting of PEG 100, PEG 200, PEG 300, PEG 400, PEG 600, PEG 800, and PEG 1,000.

9. The formulation of claim 1, wherein the PEG is PEG 400.

10. The formulation of claim 1, wherein the PEG is in an amount between about 0.01% w/v and about 1% w/v, inclusive.

11. The formulation of claim 1, wherein the PEG is in an amount of about 0.4% w/v.

12. The formulation of claim 1 comprising poloxamer F127, PEG400 and borate buffer, formulated with or without a preservative.

13. The formulation of claim 12 comprising about 12% poloxamer F127. 0.4% PEG400 and 1 mM borate buffer, formulated with or without a preservative such as 0.01% benzalkonium chloride (“BAK”), at either 72 mOsm or 150 mOsm.

14. The formulation of claim 1, wherein the formulation releases the therapeutic, prophylactic, or diagnostic agent at the mucosal or epithelial surface over a period of at least one hour.

15. The formulation of claim 14, wherein the formulation releases the therapeutic, prophylactic, or diagnostic agent at the mucosal or epithelial surface over a period of at least 24 hours.

16. The formulation of claim 1, wherein the gel-forming polymer forms a uniformly thick layer at the time of administration onto the mucosal or epithelial surface.

18. The formulation of claim 1, wherein the mucosal or epithelial surface is ocular surface of the eye.

19. The formulation of claim 18, wherein the formulation retains an effective concentration of a therapeutic, prophylactic, or diagnostic agent at the epithelial tissues inside the eye for more than a week.

20. The formulation of claim 19, wherein the epithelial tissue inside the eye is selected from the group consisting of cornea, aqueous humor, sclera, conjunctiva, iris, lens, retina, and retinal pigment epithelium.

21. The formulation of claim 1 for administration in the form of a dry powder, gel, or liquid, preferably eye drops.

22. The formulation of claim 21, wherein the formulation is provided in a single or multiple dosage unit for administration.

23. The formulation of claim 1, comprising a therapeutic, prophylactic, nutraceutical, or diagnostic agent,
wherein the agent is a protein or peptide, small molecule, sugar or polysaccharide, lipid, glycolipid, glycoprotein, nucleic acid, oligomer or polymer thereof, or small molecule.

24. The formulation of claim 1, wherein the agent is selected from the group consisting of steroids, glaucoma agents, tyrosine kinase inhibitors, immunosuppressive agents, anti-fibrotic agents, anti-infectives, antioxidants that prevent oxidative and/or nitrosative damage, hormones and chemotherapeutic agents.

25. A method for administering an agent to a mucosal or epithelial surface of a subject comprising administrating the formulation of claim 1 to a mucosal or epithelial surface in need thereof in the subject.

26. The method of claim 25, wherein the mucosal or epithelial surface is an ocular surface of the eye.

27. The method of claim 26, wherein the method treats or prevents one or more diseases or disorders of the eye.

28. The method of claim 27, wherein the disease or disorder is selected from the group consisting of glaucoma, dry eye syndrome (DES), macular degeneration, diabetic retinopathy, scleroderma, and cancer.

29. The method of claim 26, wherein the formulation is administered as an eye drop into the eye of the subject, wherein the formulation forms a gel at the surface of the eye having a thickness of between about 0.01 mm and 2 mm, inclusive gel at the surface of the eye preferably having a tonicity of between about 50 mOsm/L and about 280 mOsm/L, inclusive.
